(19) United States
(12) Patent Application Publication
(10) Pub. No.: US 2023/0285714 A1
(43) Pub. Date: Sep. 14, 2023

(54) Flow Indwelling Urinary Catheter

(71) Applicant: Mohamed Hussein Higgy, Redwood City, CA (US)

(72) Inventor: Mohamed Hussein Higgy, Redwood City, CA (US)

(21) Appl. No.: 18/110,735

(22) Filed: Feb. 23, 2023

Related U.S. Application Data
(63) Continuation-in-part of application No. 16/859,747, filed on Apr. 27, 2020, now abandoned.

(51) Int. Cl. A61M 25/00

(52) U.S. Cl. CPC A61M 25/0017; A61M 25/0026; A61M 2210/1085

(57) ABSTRACT

Improved flow indwelling urinary catheters are disclosed in which inflow openings at a distal end of a catheter are distally biased and in proximity to a distal end of a catheter balloon resulting in greater urine outflow volume and velocity, greater urine emptying volume from the bladder and patent urine outflow as the catheter is withdrawn from the bladder.

### CROSS-REFERENCE TO RELATED APPLICATIONS

This non-provisional application is a Continuation-in-Part of U.S. patent Ser. No. 16/859,747 filed Apr. 27, 2020, the entire disclosure of which is incorporated herein by reference.

### FIELD OF THE INVENTION

The present invention pertains generally to urethral catheters and more particularly to improved flow indwelling urinary catheters.

### BACKGROUND OF THE INVENTION

Urinary catheters have been available and used for decades to facilitate draining urine from a bladder in situations where a person is incontinent, has urethral obstructions, such as strictures, or to assist in urine drainage where the bladder muscle is, not functioning normally. Indwelling urinary catheters are typically delivered through the urethra and into the bladder. A distal end of the catheter is positioned above the bladder neck and a small balloon is inflated to retain the catheter in the bladder. Conventional urinary catheters, such as a Foley catheter, have become ubiquitous in the art and, typically, have a distal end with flow ports passing through side walls of the distal end of the catheter. These flow ports are typically positioned proximal to a distal tip of the catheter, leaving a relatively large distal portion of the catheter and typically have openings that are oriented perpendicular to the longitudinal axis of a flow lumen in the catheter with which the openings communicate.

A major shortcoming of conventional indwelling urinary catheters is that the inflow openings are either positioned a relatively great distance distally from the balloon and open perpendicular to the longitudinal axis of the drainage lumen in the catheter. This configuration leads to inefficient bladder drainage and occlusion of the inflow openings by the urethral walls as the catheter is being withdrawn from the bladder.

Mikhail et al. (U.S. Pat. No. 6,050,934) have disclosed an indwelling urinary catheter having a palpitatable multi-axial dome-type discharge valve with protective shoulders. The protective shoulders are raised above other unprotected portions of an outer surface of the valve. The valve wall is made substantially of a first material and each of the protective shoulders include an elongated thickness of a second material which is more rigid than the first material. The valve has a peripheral trough to maximize drainage. Valve openings traverse an arcuate pathway, and adjacent valve elements are separated by an intermediate rib to ensure a reliable closure. The catheter body in Mikhail et al. defines a central lumen extending from and communicating with Murphy Eye openings through the side wall of the catheter body.

However, the Murphy Eye openings in Mikhail et al. have a serious defect. Urine inflow ceases or dramatically slows down as soon the Murphy Eye openings pass into the bladder neck a short distance and are occluded by either the bladder neck tissue or the urethral tissue.

Vega (U.S. Pat. No. 4,249,536) teaches a flexible retention catheter that includes a soft, pliable cone-shaped tip with a reduced urethral contact surface defined by spiral grooves or hair-like projections. A magneto-insert embedded within the tip is used in cooperation with an electromagnet externally of the catheter to propel the catheter along the urethra by repulsive or attractive lines of magnetic force. The tip of the catheter is partially divided along one transverse line to form a hinged tip segment. The tip portion may also be divided longitudinally along a line intersecting with a transverse dividing line forming two hinged tip segments. The segments are releasably joined by releasable fasteners. The hinged tip segments are swung about the hinge connection against the inflated surface of the balloon by tension strips that extend along the balloon and move in response to a distention thereof to expose the drainage lumen within the tip. Other string actuators also traverse the balloon and elastically spread diametrically-divided end segments of a portion of the lumen to retain a segment of the catheter in situ in the urethra while the remaining portion of the lumen disjoined therefrom is withdrawn from the urethra. The implanted portion of the catheter is adjoined with an inflation lumen that extends along the length of the urethra. A collapsible sheath is adhered to the implanted segment in a fluid-conducting relation with the drainage lumen thereof.

Vega has designed drainage eyelets extending in the side wall of the tip to drainage lumen. However, the drainage eyelets in have the same a serious defect. Urine inflow ceases or dramatically slows down as soon the drainage eyelets pass into the bladder neck a short distance and are occluded by either the bladder neck tissue or the urethral tissue.

A recent attempt to address some of these disadvantages of the Foley catheter has been undertaken by The Flume Catheter Company (Suffolk, Great Britain) as exemplified in U.S. Pat. No. 10,195,394. The urine inflow openings of the FLUME catheter are positioned much closer to the proximal end of a channeled balloon than is found in the Foley catheter and some distance from the distal tip of the catheter. This position of the urine inflow openings, however, has been found to become easy occluded as the balloon is deflated for withdrawal or either or both of the balloon channels is not clear to allow urine flow to the urine inflow opening. Both of these situations lead to disadvantageous cessation of urine flow and inadequate bladder emptying.

### SUMMARY OF THE INVENTION

It is an objective of the present invention to provide an indwelling urinary catheter in which inflow openings communicating with a catheter flow lumen are positioned a relatively minor distance distally from a catheter balloon.

It is a further object of the present invention to provide an indwelling urinary catheter in which the inflow openings are oriented on a distal facing bias and non-perpendicular relative to the longitudinal axis of the flow lumen.

It is a still further objective of the present invention to provide an indwelling urinary catheter having a generally conical distal tip, wherein the inflow openings are in the generally conical distal tip.

It is another objective of the present invention to provide an indwelling urinary catheter having a primary lumen for urine drainage and a secondary lumen for balloon inflation.

It is yet a further objective of the present invention to provide an indwelling urinary catheter having a tertiary lumen for delivery or withdrawal of fluids, such as drugs, that extends from a proximal end of the indwelling urinary catheter and open at a distal end of the indwelling urinary catheter.

It is yet a still further objective of the present invention to provide an indwelling urinary catheter that maintains patency of fluid flow into the flow lumen as the balloon is deflated and the catheter is withdrawn through the urethra.

It is yet another objective of the present invention to provide an indwelling urinary catheter that is configured to allow an increased volume of urine drainage from the bladder when it is placed in the bladder.

It is a further objective of the present invention to provide an indwelling urinary catheter that is configured to allow for an increased rate of urine drainage from the bladder when it is placed in the bladder.

It is still another objective of the present invention to provide an indwelling urinary catheter having a balloon with a shape and inflation capacity that is configured to decrease the bladder insertion distance of the catheter to achieve urine drainage from the bladder.

It is yet still another objective of the present invention to provide an indwelling urinary catheter wherein the balloon shape, inflation capacity, and positioning of the urine inflow openings of the urinary catheter are configured to increase the urine drainage volume and drainage velocity from the bladder, while increasing patient comfort while the urinary catheter is in place in the bladder.

It is still another objective of the present invention to provide a method of draining urine from a urinary bladder in which the urinary bladder is at least substantially emptied of urine and without appreciable urine load remaining within the bladder.

It is yet another objective of the present invention to provide a method of draining urine from a urinary bladder that includes the steps of placing the inventive indwelling urinary catheter within a urinary bladder through the urethra, inflating the balloon thereby securing the indwelling urinary catheter in the bladder and positioning the urine inflow openings superior to and adjacent a distal of the balloon, draining urine through the urine inflow openings and into a urine drainage lumen within the indwelling urinary catheter, and upon removal of the indwelling urinary catheter, maintaining urine flow into the urine inflow openings during removal of the indwelling urinary catheter to at least substantially empty the urinary bladder.

A feature of the present invention is that the catheter allows a continuance flow of urine during the catheter is withdraw in the urethra.

Another feature of the present invention is that the intra urethral leakage can be reduced or eliminated by reducing the balloon inflated volume.

The above features and advantages and other features and advantages of the present invention are readily apparent from the following detailed description of the best modes for carrying out the invention when taken in connection with the accompanying drawings.

### BRIEF DESCRIPTION OF THE SEVERAL VIEWS OF THE DRAWINGS

The present invention is illustrated by way of example, and not by way of limitation, in the figures of the accompanying drawings and in which like reference numerals refer to similar elements. All the figures are schematic and generally only show parts which are necessary in order to elucidate the invention. For simplicity and clarity of illustration, elements shown in the figures and discussed below have not necessarily been drawn to scale. Well-known structures and devices are shown in simplified form, omitted, or merely suggested, in order to avoid unnecessarily obscuring the present invention.

FIG. 1 is a perspective of a first embodiment of the indwelling urinary catheter in accordance with the present invention.

FIG. 2 is a side elevational view of the first embodiment of the indwelling urinary catheter in accordance with the present invention.

FIG. 2A is a cross-sectional view taken along line 2A-2A of FIG. 2.

FIG. 3 is a first side elevational view of a distal end of the indwelling urinary catheter in accordance with the present invention.

FIG. 4 is a second side elevational view of the distal end of the indwelling urinary catheter in accordance with the present invention.

FIG. 5A is a side elevational view of the distal end of the indwelling urinary catheter of the present invention illustrating a balloon in its inflated state.

FIG. 5B is a cross-sectional view taken along line 5B-5B of FIG. 5A.

FIG. 5C is a side elevational view of a distal end of a conventional indwelling urinary catheter in its inflated state.

FIG. 5D is a cross-sectional view taken along line 5D-5D of FIG. 5C.

FIG. 6 is a perspective view of a second embodiment of the indwelling urinary catheter in accordance with the present invention.

FIG. 6A is a transverse cross-sectional view taken along line 6A-6A of FIG. 6.

FIG. 7 is a fragmentary view taken along circle 7 of FIG. 6.

FIG. 8 is a diagrammatic illustration of a conventional Foley catheter placed in a bladder illustrating urine flow into the catheter.

FIG. 9 is a diagrammatic illustration of the present invention placed in a bladder illustrating urine flow into the inventive indwelling urinary catheter.

FIG. 10 is a diagrammatic illustration of a conventional Foley catheter placed in a bladder illustrating urine drainage level in the bladder.

FIG. 11 is a diagrammatic illustration of the present invention placed in a bladder illustrating urine drainage level with the inventive indwelling urinary catheter.

FIG. 12 is a diagrammatic illustration of a bladder showing occlusion of urine flow during removal of a conventional indwelling urinary catheter.

FIG. 13 is a diagrammatic illustration showing continuous urine flow during removal of the indwelling urinary catheter of the present invention.

FIG. 14 is a diagrammatic illustration showing urinary bladders and comparative urine drainage levels between a prior art Foley catheter and the indwelling urinary catheter of the present invention.

FIG. 15 is a diagrammatic side elevational view of the indwelling urinary catheter of the present invention illustrating dimensional variables.

FIG. 16 is a diagrammatic side elevational view of the indwelling urinary catheter of the present invention illustrating more dimensional variables.

FIG. 17 is a top view of the indwelling urinary catheter of the present invention illustrating some dimensional variables.

FIG. 18 is a top view of the indwelling urinary catheter of the present invention illustrating some dimensional variables.

FIG. 19 shows the interference between the balloon neck and the bladder neck when the balloon is inflated at 10 ml volume in order to solve the issue with intra urethral leakage.

FIG. 20A shows the gap allowing a consentience flow of urine when the balloon is deflated, and the catheter is traversed through the urethra.

FIG. 20B also shows the gap allowing a consentience flow of urine when the balloon is deflated, and the catheter is traversed through the urethra.

FIG. 21 is a table showing some parameters of catheters.

FIG. 22 is another table showing some parameters of catheters.

### DETAILED DESCRIPTION OF THE PREFERRED EMBODIMENT

The terminology used herein is for the purpose of describing example embodiments only and is not intended to be limiting. For purposes of clarity, the following terms used in this patent application will have the following meanings:

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

When an element or layer is referred to as being “on,” “engaged,” “connected,” or “coupled” to or with another element, it may be directly on, engaged, connected or coupled to the other element or layer, or intervening elements or layers may be present. In contrast, when an element is referred to as being “directly on,” “directly engaged to,” “directly connected to,” or “directly coupled to” or with another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

“Substantially” is intended to mean a quantity, property, or value that is present to a great or significant extent and less than, more than or equal to totally. For example, substantially vertical may be less than, greater than, or equal to completely vertical.

“About” is intended to mean a quantity, property, or value that is present at ±10%. Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range, including endpoints given for the ranges.

Recitation of ranges of values herein are merely intended to serve as a shorthand method of referring individually to each separate value falling within the recited range, unless otherwise indicated herein, and each separate value is incorporated into the specification as if it were individually recited herein.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, biomedical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

This detailed description of exemplary embodiments makes reference to the accompanying drawings, which show exemplary embodiments by way of illustration. While these exemplary embodiments are described in sufficient detail to enable those skilled in the art to practice the disclosure, it should be understood that other embodiments may be realized and that logical changes and adaptations in design and construction may be made in accordance with this disclosure and the teachings herein without departing from the spirit and scope of the disclosure. Thus, the detailed description herein is presented for purposes of illustration only and not of limitation.

The present invention includes both an indwelling urinary catheter 10 and a method of at least substantially emptying a urine load from a urinary bladder without an appreciable urine load remaining within the bladder. In accordance with the method of the present invention, the method of draining urine from a urinary bladder includes the steps of placing the inventive indwelling urinary catheter within a urinary bladder through the urethra, inflating the balloon thereby securing the indwelling urinary catheter in the bladder and positioning the urine inflow openings superior to and adjacent a distal of the balloon, draining urine through the urine inflow openings and into a urine drainage lumen within the indwelling urinary catheter, and upon removal of the indwelling urinary catheter, maintaining urine flow into the urine inflow openings during removal of the indwelling urinary catheter to at least substantially empty the urinary bladder.

Turning now to the accompanying Figures in which exemplary embodiments of the present invention are illustrated and common reference numerals denote common features among the exemplary embodiments. Two main embodiments of the urinary catheter of present invention are illustrated in the accompanying Figures. A first embodiment is a two-way catheter 10, having a urine flow lumen and a balloon inflation lumen and a second embodiment is a three-way catheter 30, having three lumens, including a urine flow lumen, a balloon inflation lumen, and a tertiary lumen.

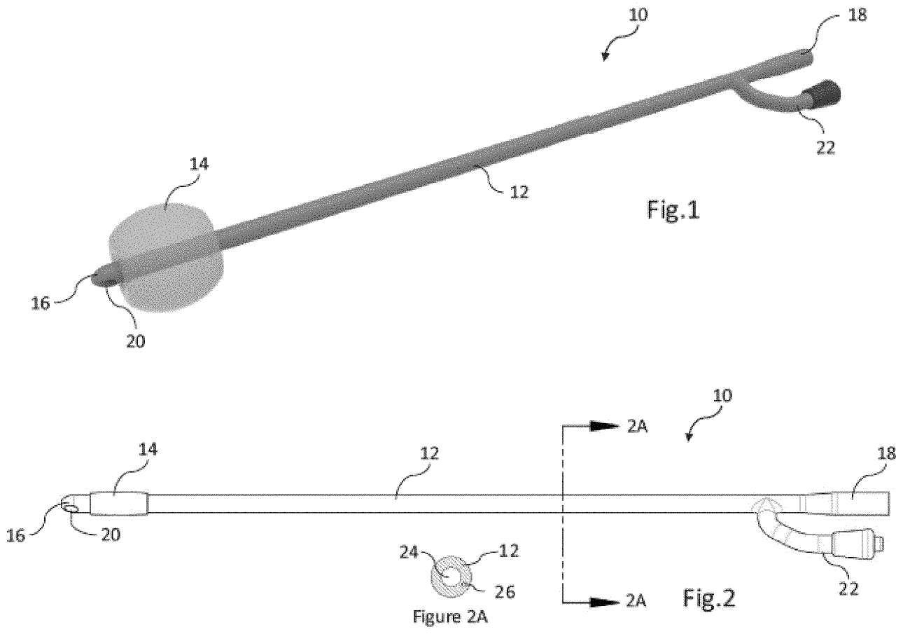

As illustrated in FIGS. 1-4, catheter 10 is a single unitary construct. It is consisted generally of two-way catheter having catheter body 12 which is an elongate tubular member having a drainage lumen 24 and the balloon inflation lumen 26 (See, FIG. 2A), extending through the catheter body 12. A balloon 14, is affixed to the catheter body 12 at a distal portion thereof. The balloon 14 is typically concentrically affixed to the catheter body 12 and inflates in a substantially toroidal spherical shape as a fluid, typically saline, is injected into the balloon 14. The balloon inflation lumen 26 is in fluid flow communication with balloon 14 and with an inflation connector 22 positioned at a proximal end of the catheter body 12. Typically, urinary catheter balloons 14 have a maximum balloon inflation capacity between about 1.5 ml to about 35 ml, where smaller balloons are employed in pediatric applications and larger balloons are employed in adults.

A drainage port 18 is in fluid flow communication with the drainage lumen 24, shown in FIG. 2A, and is configured to accommodate coupling to a urine drainage line at a proximal end of the catheter body 12. At least one, and preferably at least two, inflow openings 20 are provided at a distal tip 16 of the catheter body 12. The inflow openings 20 communicate with the drainage lumen 24 at a distal portion of the catheter body 12. The inflow openings 20 have a distal facing bias relative to the longitudinal axis 13 of the catheter body 12. The distal tip 16 of the catheter body 12 preferably has a substantially conical or frustoconical shape in the inflow openings 20 are positioned in diametric opposition relative to each other in the distal tip 16.

While the inflow openings 20 may have a wide variety of transverse opening shapes in the plane of the distal tip 20, to maximize the open surface area of each of the inflow openings 20, the inflow openings 20 preferably have a transverse opening shape that conforms to the conical shape of the distal tip 16. The transverse opening shape of the inflow openings 20 may be substantially circular, elliptical, ovoidal, triangular, or the like that is configured to maximize the open area and urine inflow into the drainage lumen 24. In this manner, the open surface area of the inflow openings 20 at the surface of the distal tip 16 is configured to allow the greatest opening configuration to permit urine inflow into the inflow openings 20.

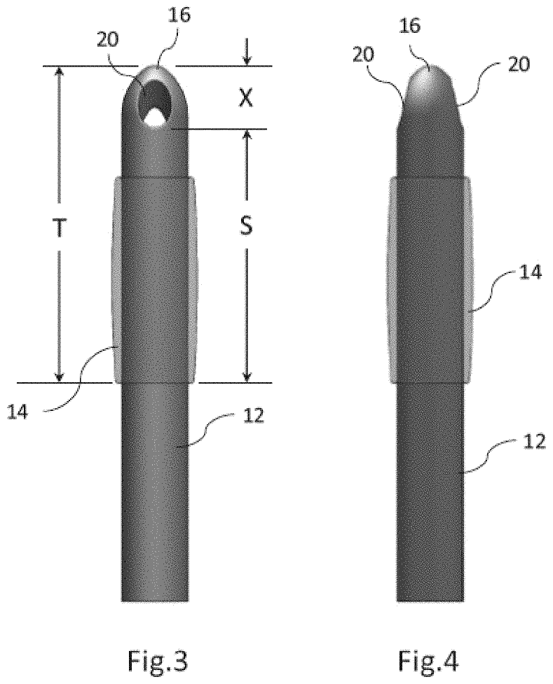

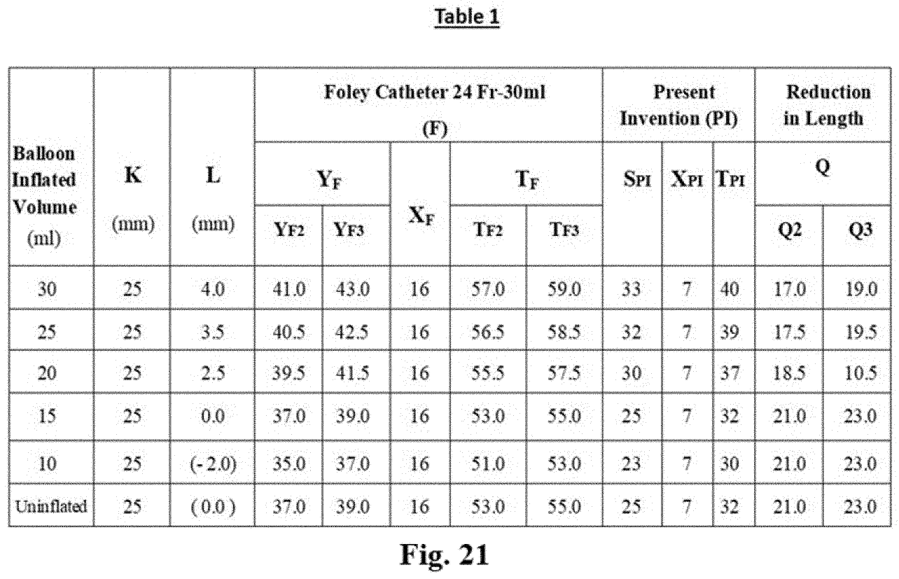

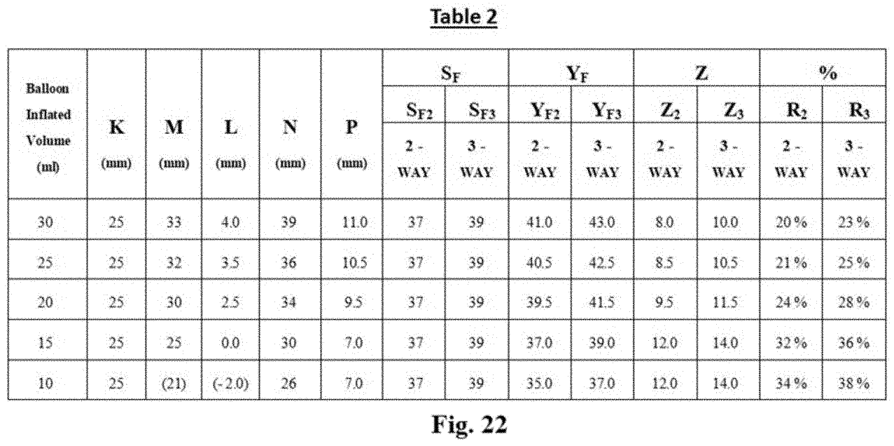

With particular reference to FIG. 3, there is shown the distal portion of indwelling urinary catheter 10 in which the balloon 14 is deflated. The positioning of the inflow openings 20 in the distal tip 16 relative to the balloon 14 is significant in the inventive urinary catheter 10. Distance S is the measurement taken from a proximal end of uninflated balloon 14 to a proximal end of inflow opening 20. Distance T is the measurement taken from the proximal end of uninflated balloon 14 to the distal end of distal tip 16. It will be understood, therefore, that the distance X between the proximal end of inflow opening 20 and the distal end of distal tip 16 is expressed by the equation: T_{F}=S_{F}+X_{F}=(Y_{F}−L+X_{F}). This equation is applied to Foley catheter, where the values of S_{F} are shown in table 2, FIG. 22, at a different balloon inflated volume. The following data in related to the present invention, that show T_{PI}=S_{PI}+X_{PI}=(K+2L+X_{PI}), therefore X_{PI}=T_{PI}−S_{PI} or X_{PI}=T_{PI}−(K+2L), where Q is the difference in values between the value of T_{F} for Foley catheter and T_{PI} for the present invention as are shown in table 1, FIG. 21.

The “X” dimension is determined the distance between bottom end of the inflow opening to the tip end of the catheter, and also the length of the conical shape. This dimension is variable and depends on the size of the drainage lumen. This is a design dimension for size 24 Fr catheter, that will be able to accommodate the size of the two inflow openings, which each one of them should have a cross section opening area is equal or greater than the cross-section area of the drainage lumen. Also this dimension was used as constant for guidance and to compare between the new design and Foley catheter. It is also used to shorten the neck of the catheter in order to almost empty the bladder of urine all the time and give the patient much more of comfort. None of the prior arts is determined the length of the conical shape.

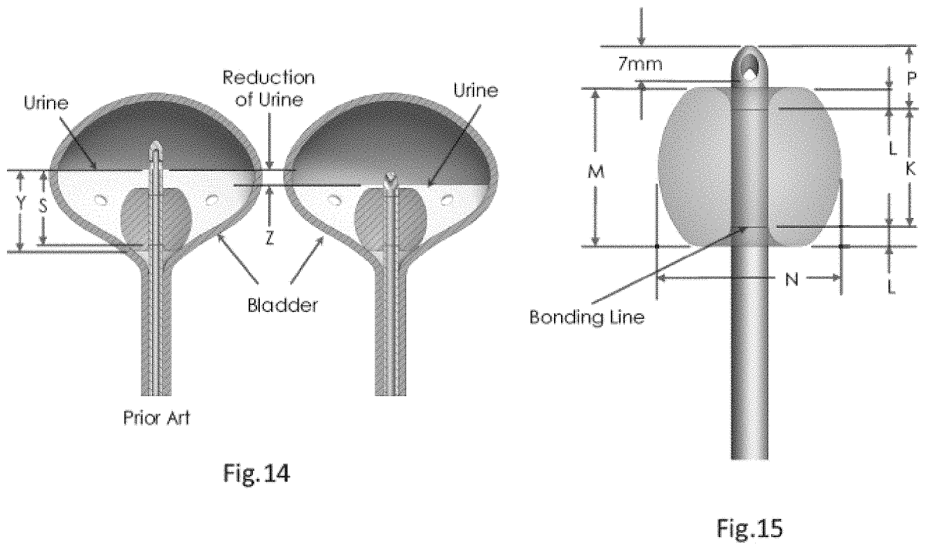

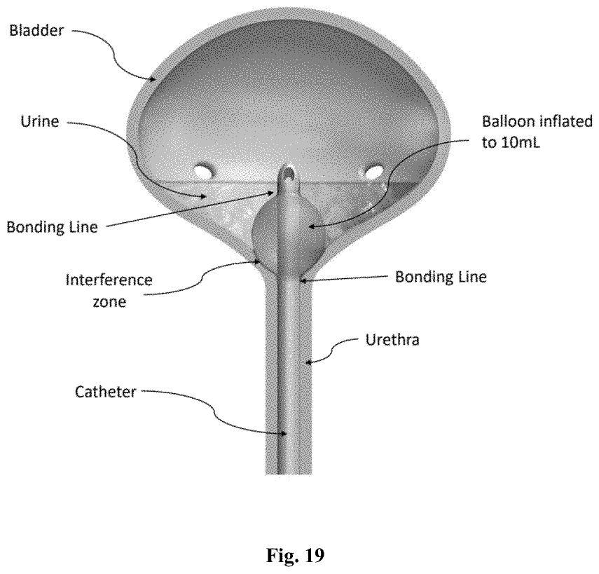

Brief Summary of Table 1 as shown in FIG. 21 Source of information are from FIG. 3, FIG. 14. FIG. 15, FIG. 19 and table 2, FIG. 22.

First condition: Balloon Deflated
A—Foley Catheter (F); S=S_{F} & Y_{F}=S_{F} & T_{F}=S_{F}+X_{F};
B—Present Invention (PI); S=K & T_{PI}=K+X_{PI}
The reduction in length Q=T_{F}−T_{PI}

### Second Condition: Balloon Inflated at Different Inflated Volumes;

A—Foley Catheter (F); S=S_{F} & Y_{F}=S_{F}+L & T_{F}=Y_{F}+X_{F}
B—Present Invention (PI):

S_{PI}=M=K+2L & T_{PI}=S_{PI}+X_{PI}
The reduction in length Q=T_{F}—T_{PI}
Therefore; X_{PI}=T_{PI}−S_{PI} OR X_{PI}=T_{PI}−(K+2L) OR X_{PI}=T_{PI}−M
Verification of value at 30 ml

X_{PI}=T_{PI}−S_{PI}=40−33=7 mm
Verification of value at 10 ml

X_{PI}=T_{PI}−S_{PI}=30−23=7 mm
Verification of value at 30 ml

X_{PI}=T_{PI}−(K+2L)=40−(25+2(4)=40−33=7 mm
Verification of value at 10 ml
X_{PI}=T_{PI}−(K+2L), S_{PI} in this case is equal to (K+L) due to one L is burred inside the bladder neck. Therefore in this case Xₚᵢ=T_{PI}−(K+L)

X_{PI}=T_{PI}−(K+L)=30−(25+(−2))=30−23=7 mm

The above equations can be used for all balloon inflated volumes settings, except at 10 ml balloon inflated volume, In this case the inflated potion of “M” is measured 21 mm and “K” as a fixed value=25 mm. Since M=K+2L Therefore 2L=M—K=(21−25)=−4 mm. and L=−2 mm
The distance between the bottom of the balloon to the bottom of the inflow opening is S_{PI}. Hence (S_{PI}=M=K+2 L), one of the “L” value is burred inside the bladder neck as it is shown in FIG. 19, therefore S_{PI}=K+L=25+(−2). S_{PI}=23 mm,

S_{PI}=M as the inflated potion plus the top portion of the uninflated (21+2)=23 mm

T_{PI}=S_{PI}+X_{PI}=32+7)=30 mm, and X_{PI}=T_{PI}−S_{PI}=30−23=7 mm

Or X_{PI}=T_{PI}−K+L=30−(25+(−2))=7 mm

In summary: The following data are used only at 10 ml balloon inflated volume;
K=25 mm. L=−2 mm, S_{PI}=K+L=23 mm, the actual expose value of M is 21+2=23 mm
While at 30 ml balloon inflated volume S_{PI}=M=(K+2 L)=33=(25+2(4)=33 mm

T_{PI}=S_{PI}+X_{PI}=33+7)=40 mm, and X_{PI}=T_{PI}−S_{PI}=40−33=7 mm

Where Y_{F2}, T_{F2}, and Q₂ are related to 2-way catheter, and Y_{F3}, T_{F3}, and Q₃ are related to 3-way catheter.

As shown in table 1, FIG. 21, when compared to a standard urinary catheter such as a Foley catheter, the values of each of T, S, and X, are all less than that found in a standard urinary catheter. As an example, in a standard Foley catheter, distance T_{F} is typically about 51 mm for a two-way catheter whereas it is about 59 mm for a three-way Foley catheter at a different balloon inflates volumes, whereas in the present invention, as illustrated distance T_{PI} is between about 30 to about 40 mm depending on the balloon volume. Further, in a standard Foley catheter, distance S_{F} is typically about 35 mm for the two-way catheter and 39 mm for the three-way catheter, whereas in the present invention distance S_{PI} is between about 23 to about 33 mm, again depending on balloon volume. Following the above equation, the distance X_{F}, between the proximal end of the flow opening and the distal end of the distal tip, is about 16 mm in a standard Foley catheter and X_{PI} is about 7 mm in the urinary catheters 10 and 30 of the present invention. The foregoing dimensions are with reference to an inflated and uninflated balloon conditions. It will be understood that when the balloon is inflated in the bladder, the proximal end of the balloon will extend between about 0 to 4 mm further depending upon the inflated balloon volume. Inflated balloon volumes may range from 10 ml to 30 ml, as shown in Table 2, FIG. 22 below.

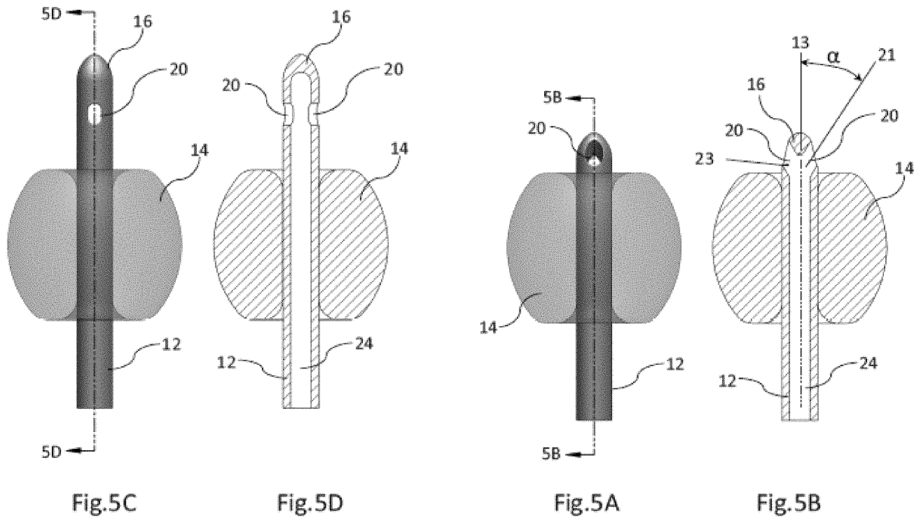

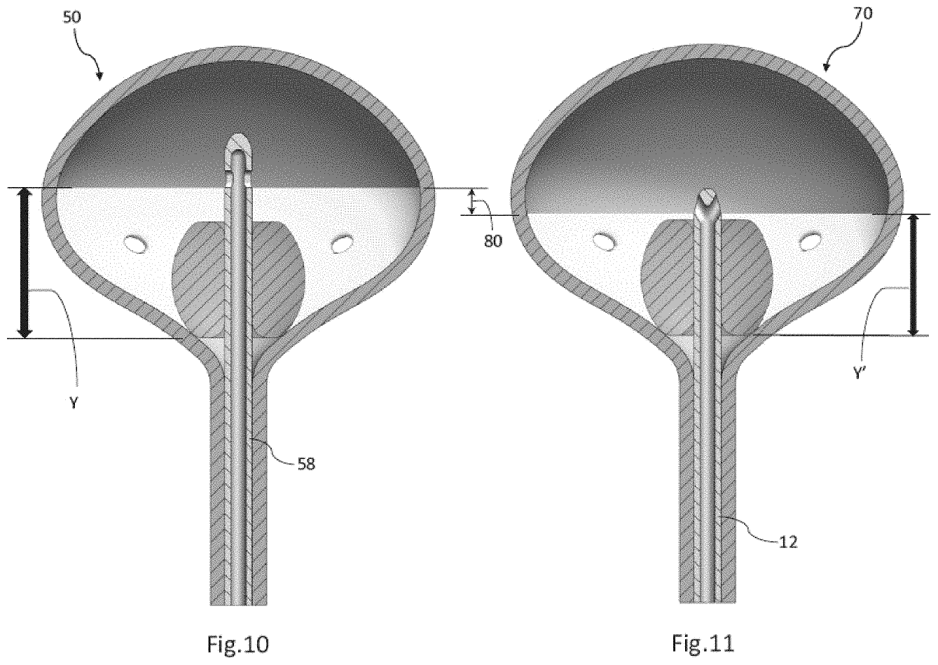

As noted above, each of the inflow openings 20 has a distal facing bias. Each of the inflow openings 20 communicate with a fluid conduit 23, which, in turn, communicates with the drainage lumen 24 of catheter body 12. As shown in FIGS. 10 and 5B, the distal bias 21 of the inflow openings 20 and the fluid conduit 23 forms angle α relative to the longitudinal axis 13 of the catheter body 12. Angle α is between about 15 to about 75° relative to the longitudinal axis 13 of the catheter body 12, preferably angle α is between about 20 to about 45° and most preferably it is between about 25 to about 35°. It will be understood that angle α of the inflow openings 20 affects the urine flow profile into and through the inflow openings 20 and by having 15 the inflow openings 20 with a distal bias of angle α, the inflow openings 20 facilitate optimal flow of urine from the bladder and into the drainage lumen 24 of catheter body 12.

It will be understood by those skilled in the art, therefore, that with both the two-way and three-way variants the indwelling urinary catheter 10, 30, respectively, of the present invention, the distal tip 16 and the inflow openings 20 are in far greater proximity to a distal end of balloon 14 than is found in conventional urinary catheters. This relatively closer approximation between the balloon 14 and the distal tip 16, positions the inflow openings 20 closer to the bladder neck relative to standard urinary catheters when the urinary catheter is properly positioned within the bladder. FIGS. 5A and 5B more clearly illustrate the proximity between the distal end of balloon 14 and the inflow openings 20 in the distal tip 16 when the balloon 14 is in its expanded state. It will be understood that the proximal end of at least one of the inflow openings 20 is positioned at substantially at the same level as or below the distal end of balloon 14. FIGS. 5C and 5D illustrate a conventional urinary catheter and the greater distance S, defined above, between the distal end of the inflated balloon 14 and the proximal end of inflow opening 20 than that found in the indwelling urinary catheter 10 of the present invention. As is shown in FIGS. 5C and 5D, in a conventional urinary catheter, the proximal end of the inflow openings 20 is distally spaced and not substantially at the same level as the distal end of balloon 14.

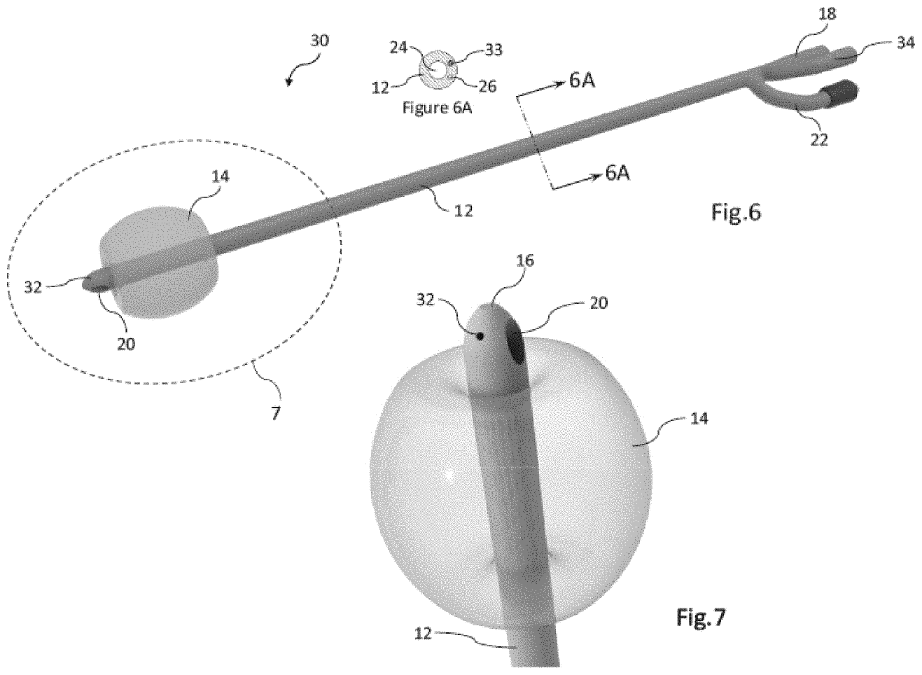

A second embodiment of the indwelling urinary catheter 30 is a single unitary construct. It is illustrated in FIG. 6. Indwelling urinary catheter 30, which is a three-way catheter, is substantially identical to indwelling urinary catheter 10, with the exception that a tertiary lumen 33 is provided, which may be employed to deliver or withdraw fluids, such as drugs or urine samples, respectively. The tertiary lumen 33 passes along the longitudinal axis of the catheter body 12 and communicates with a fluid port 32 at the distal end of the tertiary lumen 33 and with a fluid connector 34 at a proximal end of the catheter body 12. One skilled in the art will understand that the tertiary lumen 33 is similar to the inflation lumen 26 in FIG. 2A and extends from the fluid port 32 to the fluid connector 34. The fluid port 32 passes through the distal tip 16 and is positioned between the inflow openings 20. Fluid port 32, tertiary lumen 33 and fluid connector 34 are configured to allow a syringe or other vessel to be coupled to the fluid connector 34 and deliver a pharmaceutically active agent into and through the tertiary lumen 33 and out the fluid port 32 and into the bladder. Similarly, fluids, such as urine samples, may be withdrawn through the fluid port. The term “connector” is intended to encompass different mechanical or other types of engagement configurations that allow for a syringe or other vessel to be fluidly coupled to the tertiary lumen 33 to deliver a fluid from the syringe or other vessel to the tertiary lumen 33. Examples of suitable connectors include a luer fitting, a friction fit connector, a snap fit, a one-way valve, or the like. FIG. 7 is an enlarged view along circle 7 of FIG. 6 and more clearly illustrates the positioning of the fluid port 32 in the distal tip 16 and relative to the inflow opening 20.

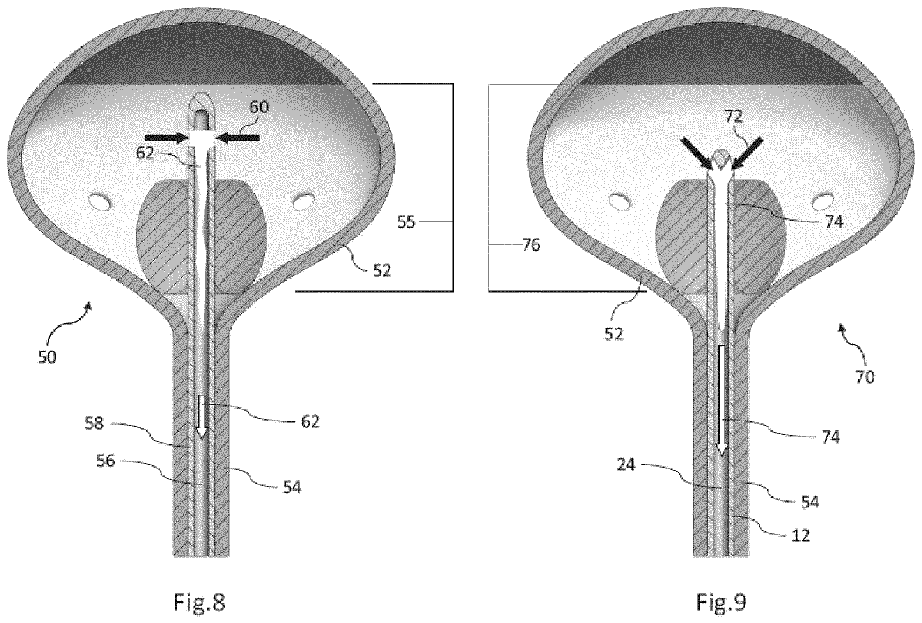

FIGS. 8-13 depict comparative differences between standard urinary catheters and the inventive indwelling urinary catheters 10, 30 in urine inflow vector into the inflow openings 20, the relative velocity of urine flow into and through the flow lumen, the resulting levels of urine remaining in the urinary bladder, and urine flow as the respective catheters are removed through the urethra.

FIG. 8 depicts a urine loaded bladder 50 with a standard indwelling urinary catheter 58, such as a Foley catheter, in its deployed state 50 in a urinary bladder 52 and passing through the bladder neck 54. The urine load 55 with the standard indwelling urinary catheter 58 is depicted in FIG. 8. It will be noted that the positioning of the inflow openings on lateral wall surfaces of catheter 58 are positioned toward an upper portion of the urine load 55 and positioned to create a urine inflow vector 60 that is substantially perpendicular to the longitudinal axis of catheter 58. This results in a mostly moderate flow of urine 62 into the inflow openings and through drainage lumen 56 of catheter 58.

In contradistinction to the standard indwelling urinary catheter 58, as illustrated in FIG. 9, illustrating a urine loaded bladder 70 with the inventive indwelling urinary catheter 12, when placed in bladder 52 through the bladder neck 54 has an inflow vector 72 through the inflow openings 20 that is angularly displaced between perpendicular to and parallel with the longitudinal axis of indwelling urinary catheter 12. The urine inflow openings 20 are positioned far closer to the distal end of the balloon than in the standard indwelling urinary catheter 58. The angular displacement of urine inflow vector 72 is between about 15 to about 75° and has a distal bias relative to the longitudinal axis of the indwelling urinary catheter. In this configuration, the urine flow from urine load 76 into and through the distal openings 20 forms a substantially continuous flow of urine into and through the drainage lumen 24 of indwelling urinary catheter 12. It will be appreciated that the velocity of urine flow 74 in a continuous robust flow circuit, such as that provided by the present invention, is greater than the moderate velocity of urine flow, as is found in standard indwelling urinary catheters.

In both the standard urinary catheters and the inventive urinary catheters, once the urine level in the bladder reaches the proximal end of the urine inflow openings 20, the urine flow will drain in a drop-wise fashion. If this condition remains for a period of time, solidification or crystallization of urine in the drainage lumen may occur. For example, during patient sleep cycles, the bladder will accumulate urine and with the patient in a horizontal position, the urine load level may not reach the level of the urine inflow openings 20 such that drainage will resume. As the patient assumes an upright position, the urine load will then reach the level of the urine inflow openings 20 and begin draining into the drainage lumen 24 A higher urine load level inside the bladder will result in a greater urine drainage flow velocity in the present invention, which will aid in flushing any solids or crystalline substances that may accumulate in the drainage lumen 24.

FIGS. 10, 11 and 14 depict a comparison of urine drainage between the standard urinary catheter 58 in a bladder 50 and the inventive urinary catheter 12 in a bladder 70, respectively, as measured by the level of urine, Y and Y′, remaining in the urinary bladder upon maximal emptying for each catheter. As will be seen in FIG. 10, because of the relatively greater distance between the proximal end of the balloon and the proximal end of inflow opening 20, urine in the bladder will only drain to a distance Y, leaving a relatively large volume of urine load in the bladder. In contrast, as illustrated in FIG. 11, the relatively closer proximity between the proximal end of balloon 14 and the proximal end of inflow openings 20, allows for a greater volume of urine drainage from the bladder and a corresponding smaller volume of urine load, as represented by Y′, remaining in the bladder. The difference between the remaining urine load with the standard urinary catheter 58 and the remaining urine load with the inventive urinary catheter 12 is represented by reference numeral 80 denoted between FIGS. 10 and 11.

The same comparison is illustrated in FIG. 14, illustrating the difference Z between the remaining urine load in a standard urinary catheter 58 and that in the inventive urinary catheter 12 when drained to the level of the proximal end of urine inflow opening 20, respectively. The difference Z in remaining urine load is also influenced by balloon inflation volume as is illustrated in Table 2, FIG. 22 below.

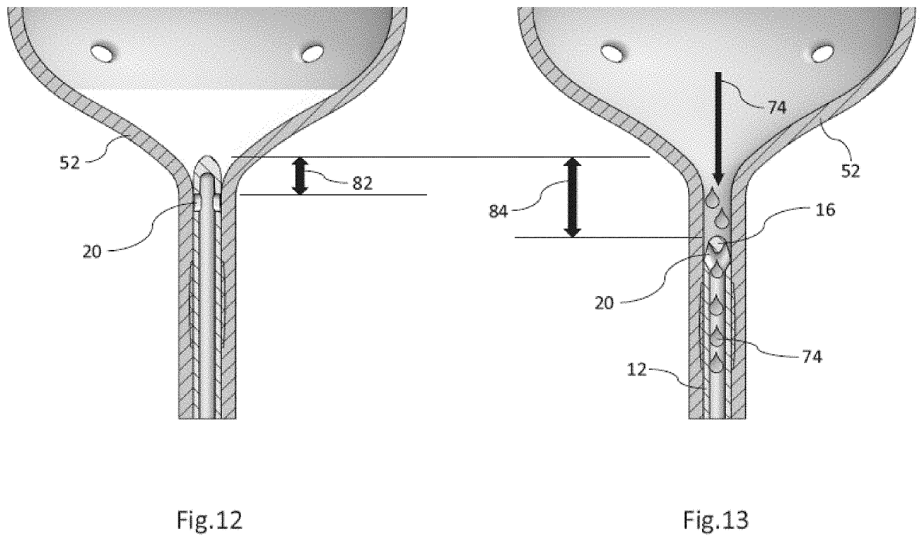

FIGS. 12 and 13 also illustrate a comparison between a standard indwelling urinary catheter 58 and the inventive indwelling urinary catheter 10, 30, respectively, during catheter withdrawal. In the standard indwelling urinary catheter 58, urine inflow ceases as soon the inflow openings 20 pass into the bladder neck a short distance 82 and are occluded by either the bladder neck tissue or the urethral tissue. In stark contrast, the distally biased orientation of the inflow openings 20 in the inventive indwelling urinary catheter 12, causes the inflow openings 20 to remain open and not become occluded by either the bladder neck or urethra as the catheter is being withdrawn even a relatively large distance 84, as compared to the standard indwelling urinary catheter 58. In this manner, urine flow 74 from the bladder and into the inventive indwelling urinary catheter 10, 30, remains continuous until either the muscles of the bladder or the bladder neck contract to seal off the bladder from the urethra.

Thus, with respect to the Foley catheter, the present invention is advantageous in reducing the total length of the catheter inside the bladder. In one example of the embodiments of the invention, using the equation Q=T_{F}−T_{PI}. it can be calculated the catheter is capable of reducing the total catheter length inside the bladder by 17 mm for the two-way embodiment and 23 mm for the three-way embodiment when compared with a standard Foley catheter at different balloon inflated volume, as it shown in table 1, FIG. 21. Values of L and M are shown in Table 2, FIG. 22 below. The risk of damage to the bladder wall due to the distal tip impacting the bladder during patient movement or insertion is minimized as a result of the catheter tip being far shorter than that of a Foley catheter. The present invention increases the volume of urine drainage and, therefore, reduces the level of urine remaining in the bladder by at least 8 mm for the two-way catheter embodiment 10 and about 10 mm for the three-way embodiment 30, both relative to the Foley catheter, at 30 ml balloon inflated volume as shown in Table 2, FIG. 22.

Due to the generally ovular shape of the bladder, its widest dimension is intermediate the superior or inferior aspects of the bladder. Therefore, a reduction in the height of the urine load within the bladder does not directly correlate with the volume of urine drained from the bladder. A urine load height reduction of 1 mm at the center of the bladder is a greater reduction than a similar 1 mm urine load height reduction at the superior or inferior aspects of the bladder, both of which have smaller transverse cross-sectional areas. Based on that observation, a reduction of value of 8 mm or 10 mm in urine load height may present an empty bladder, except for residual urine around the balloon 14. Upon substantial drainage of urine from the bladder, any residual urine will have a very small volume due to its being located in the inferior aspect of the bladder. The height of a residual urine load in the bladder will depend upon the balloon capacity, which should not exceed 33 mm.

Under normal conditions, the bladder's muscular contractions will occur when the micturition response occurs, typically when the bladder is full and there is a need for urination. In this case, the muscular contractions of the bladder will force urine down into the urethral opening for drainage and an empty bladder. Where an indwelling urinary catheter is placed in the bladder, the patients discomfort occurs mostly from two factors: balloon size and a greater than normal amount of urine remaining in the bladder. These factors typically cause the urge to urinate more frequently. The muscular contraction of the bladder will raise the urine level around the balloon 14 periodically as will motion, such as walking or laying down. As these events occur, the urine will drain into the urine inflow openings 20 and drain into the urinary catheter 10. Because the bottom of the urine inflow openings 20 are positioned at substantially the same level as or below the superior balloon surface, urine will flow more readily and faster into the urine inflow openings 20. This will lead to at least substantially complete urine drainage from the bladder.

The prior art references are claiming, the proximal end of each urine inflow opening is even with or below a distal end of the balloon. But they did not give any details about the relationship between the balloon and the inflow opening. In this specification there are more specific details and clarification are provided. This specification claims, the urine inflow openings are at a distal end of the catheter body positioned such that the proximal end of each urine inflow opening is substantially even with or below a distal end of the balloon when the balloon is inflated. Table #2, FIG. 22 and FIG. 15 give more details for this relationship. The listed dimensions in columns (“M” and “N”) are showing the height and the width for the inflated balloon at a different inflated volume. The allowable distant to be below the proximal end of the inflow opening and the distal end of the balloon is listed in column “L”. The value of “L” at different balloon capacities are shown in column “L” table #2. This below distance is very important in order to remain the inflow opening open not become occluded. For example the below distance at 30 ml balloon capacity is 0.5 mm, and 1.0 mm at 25 ml balloon capacity. At about 15 ml balloon capacity, the maximum below distance will be 4.0 mm, when the proximal end of the inflow opening, the distal end of the balloon and the top boding line of the balloon are all at the same level. The value of the below distance between the proximal end of the inflow opening and the distal end of the balloon can be taken as an advantage to shorten the length of the catheter above the balloon, which will result a big reduction of the residual of urine inside the bladder.

Moreover, the configuration of the inflow openings 20 both increases the urine flow rate and carry with any of the sloughed tissues (scabs) and/or any of the blood clots from the bladder, reduces the risk of solidification and blocking the catheter, eliminates the risk of the bladder wall being sucked into the inflow openings, and allows for drainage of urine as a result of the patient's normal movements. Further, as the balloon is collapsed and the catheter is being removed from the bladder, the flow of urine into the inflow openings 20 will continue until as long as the urethra remains patent during withdrawal. Where the balloon is partially collapsed, the muscular contractions of the bladder may drive the catheter into the urethra and, in conventional urinary catheters, either the balloon or the urethral walls will occlude the urine inflow openings 20 and cause stoppage of the urine into the drainage lumen. In the present invention, urine flow is maintained despite either full or partial collapse of the balloon or mispositioning of the catheter in the urethra. Overall, the foregoing advantages of the present invention will result in easier insertion into and through the urethra and into the bladder and less patient irritation and discomfort due to the greater bladder drainage and the increased velocity of urine flow into the drainage lumen.

When compared to the FLUME catheter, the present invention advantageously maintains urine flow when the balloon is collapsed, and the catheter is being withdrawn from the bladder and urethra. Further, because the balloon of the FLUME catheter is of a relatively large size and has two recesses or channels in the exterior surface of the balloon to channel urine to the urine inflow opening, that require the balloon to be folded over the distal tip, the risk of urine flow blockage is far greater than that with the present invention, leading to a greater risk of patient discomfort and irritation.

### EXAMPLES

A study was performed to determine the impact of balloon capacity and balloon shape when inflated on the life span of the indwelling inflated balloon, the amount of urine remaining inside the bladder after drainage, balloon leakage and collapse, and patient acceptance. Five (5) samples each of silicon-elastomer coated latex Foley Catheters 24 Fr 30 ml balloon inflation capacity, manufactured by MEDLINE. Of both two-way (REF. #DYND11784) and three-way designs (REF. #DYND11804) were measured for physical dimensions of the following:

(1) Distance between two bonding lines of the balloon to the catheter; Measurement “K”; (2) Height of the inflated balloon; Measurement “M=K+2L”; (3) Extended length of the balloon beyond each bonding line; Measurement “L”; (4) Outer diameter of the inflated balloon; Measurement “N”; (5) Distance between the distal tip of the inventive catheter to the distal bonding line; Measurement “P=L+7 mm”; The dimensions in column “P” are intended to be exemplary and presented only as basic guidance to one skilled in the art to practice the present invention. (6) Distance between the bottom of the urine inflow opening to the proximal bonding balloon prior to balloon inflation; Measurement “S”; (7) Distance from the bottom of the each urine inflow opening to the proximal end of the inflated balloon at any inflation capacity in a standard Foley catheter; Measurement “Y_{F}=S_{F}+L”; (8) Distance from the proximal end of urine inflow opening 20 to the proximal end of the inflated balloon at any inflation capacity in the inventive catheter; Measurement “Y_{F}=(S_{F}+L)=(K+2L)=M” this equation is valid for all different setting of balloon inflated volume except when the balloon inflated to 10 ml volume (see section 0072 for more detail); (9) Distance from proximal end of urine inflow opening to the distal end of the distal tip in the inventive catheter; Measurement “X”; (10) Height of urine reduction inside the bladder; Measurement “Z=Y_{F}−M” this equation is valid for all different setting of balloon inflated volume except when the balloon inflated to 10 ml volume (see section 0072 for more detail); and (11) Ratio of urine reduction inside the bladder; Measurement “%=Z/Y_{F}”.

FIGS. 14 and 15 graphically illustrate each of the foregoing measurements relative to the standard Foley catheter and the inventive indwelling urinary catheter depicted in FIGS. 14 and 15. In the inventive indwelling catheter in FIG. 15, distance X_{PI} was a constant 7 mm for purposes of calculated comparisons with the standard Foley catheter depicted in FIG. 14. The 7 mm distance X_{PI} was selected (see section 0071 for more detail) and to maintain a constant distance for the urine drain openings 20 in order to reduce interference between the distal tip of the catheter and the urethra and facilitate more comfortable insertion of the catheter through the urethra and into the bladder. Table 2, FIG. 22, presents the calculated data for the above measurements as a function of different inflation volume in the Foley urinary catheters 24 Fr 30 ml inflation balloon capacity

Brief summary of table 2 as shown in FIG. 22

Source of information are from FIG. 3, FIG. 14. FIG. 15, FIG. 19 and table 1
The condition: Balloon is inflated at different volume.

### A—Foley Catheter (F)

S=S_{F} & Y_{F}=S_{F}+L

### B—Present Invention (PI)

S_{PI}=K+2L=M

Z=Y_{F}−M Equation #1

Verification of value at 30 ml

Z₂=Y_{F}−M=41−33=8

Verification of value at 10 ml

Z₂=Y_{F}−M=35−23=12

Z=S_{F}+L−M Equation #2

Verification of value at 30 ml

Z₂=S_{F}+L−M=37+4−33=8

Verification of value at 10 ml

Z₂=S_{F}+L−M=37+(−2)−23=12

Use this equation X_{PI}=T_{PI}−M Or M=T_{PI}−X_{PI}

Substitute M in this equation Z=Y_{F}−M

Z=Y_{F}−(T_{PI}−X_{PI})

Z=Y_{F}—(T_{PI}−X_{PI}) Equation #3

Verification of value at 30 ml

Z₂=Y_{F}—(T_{PI}−X_{PI})=41−(40−7)=8

Verification of value at 10 ml

Z₂=Y_{F}−(T_{PI}−X_{PI})=35−(30−7)=12

Use this equation Y_{F}=S_{F}+L

Substitute Y_{F} in this equation Z=Y_{F}−(T_{PI}−X_{PI})

Z=S_{F}+L−(T_{PI}−X_{PI})

Z=S_{F}+L−(T_{PI}−X_{PI}) Equation #4

Verification of value at 30 ml

Z₂=S_{F}±L−(T_{PI}−X_{PI})=37+4−(40−7)=8

Verification of value at 10 ml

Z₂=S_{F}+L−(T_{PI}−X_{PI})=37+(−2)−(30−7)=12

All the above four equations can be used to obtain the value of Z at any setting of the balloon inflated volumes The value of T_{PI} at 10 ml inflated balloon volume was calculated differently as explained in section 0072

The value of Z at 10 ml and 15 ml balloon inflated volume are equal (12 mm for 2-way catheter and 14 mm for the 3-way catheter), but the position of the inflow opening at 10 ml inflated balloon volume is 2 mm below the position of the inflow opening at 15 ml inflated balloon. That will reduce tremendously the residual urine inside the bladder.
Where Z2 is the designation for the 2-way catheter, and Z3 is the designation for the 3-way catheter.

At 10 ml. inflation volume, the balloon was not inflated to its full geometry as shown in FIG. 19. In this case, the height of the inflated balloon (M) was 21 mm while the height between the two bonding lines (K) was 25 mm. This resulted in the inflated balloon being 2 mm shorter from each side than the bonding line length. In that case, the final step for securing the catheter in the bladder would be to pull out the catheter 2 mm or slightly more until some kind of resistance is occurred in order to seat the balloon in the bladder neck properly, which will slightly increase the ability of the catheter to reduce the volume of the urine load within the bladder. Increased patient comfort and reduced patient irritation lending itself to a higher degree of patient compliance with the indwelling catheter and, therefore, allow the patient to have a more rapid recovery. Some clinicians prefer to use a 10 ml inflation capacity primarily to increase patent comfort.

This setting of 10 ml balloon inflated volume can be used to substitute any other setting of 30. 25. 20.15 ml in order to get the benefits of shorten the length of catheter by 2 mm inside the bladder plus increase the ability of the catheter to reduce the volume of the urine load within the bladder and increase patent comfort.

The height of urine reduction inside the bladder (Z=Y_{F}−M) this equation is valid for all different setting of balloon inflated volume except when the balloon inflated to 10 ml volume (see section 0072 for more detail); was calculated based upon the difference in distance between the bottom of the urine inflow opening in the Foley Catheter and the same measurement for the inventive catheter described herein.

Intra urethral leakage is a major issue in the catheter industry based on Mikhail et al (U.S. Pat. No. 6,050,934) Col. 1 line 41-53. The leakage phenomena, it is appear when the catheter outside diameter may not compatible to urethra inside diameter (the interference between them is insufficient). In this case during the bladder spasms and or bladder contraction some urine will leak out as a few drops or more of urine depend on the condition between the catheter outside diameter and the inside diameter of the urethra. That condition will cause a great of pain for the patient especially if the catheter was used after a surgery.

At the setting of 10 ml balloon inflated volume, the balloon is not fully inflated to its geometry, the maximum diameter of inflation will show up at the middle of the distance between the two bonding lines, then the diameter gradually will decrease to a point that shows a minor inflation at the bottom and top of the balloon (the balloon layer does not in contact with the catheter body), that will cause an slightly minor increase of the diameter at the both ends (like a soft couching). By pulling out the catheter 2 mm or slightly more until some kind of resistance is occurred, that action will help to seat the neck of the balloon inside the neck of the bladder properly. This action will create a firm interference between the bladder neck and the balloon neck. This interference will reduce or eliminate the intra urethral leakage between the urethra wall and the catheter body. See FIG. 19.

In case of the balloon inflated at higher volume, the balloon is nesting in the bottom of the bladder, but the outer parameter of the balloon will raise up the neck of the balloon 0-4 mm from reaching the neck of the bladder as shown in table 2, FIG. 22 column “L”, FIG. 15, and FIGS. 8-11. Some surgeons are using the recommended inflated volume 30 ml, or sometime use a higher inflation volume than the maximum of 35 ml in order to keep the catheter longer with the patient and by substituting for any loss of water in case of any leakage due to a deterioration of the balloon layer and/or any leakage from the inflating valve.

Inflating the balloon for the first time at 10 ml may not give a homogeneous shape (sometime the balloon is inflated on one side), therefore it is recommended, after the catheter is being inserted into the bladder. The balloon should be inflated to the maximum allowable value then deflated. Repeat this action at least twice in order to stretch the balloon layer and get a symmetrical shape along the longitudinal axis. then set the balloon to 10 ml inflated volume and secure it in place.

The Foley catheter balloon may be modified to change the distance between the two bonding lines of the balloon to the catheter (K) from 25 mm to 15 mm with the potential result in an additional approximately 6 mm of reduction in the urine level within the bladder after drainage (Z). A change in balloon length to 15 mm, together with the use of smaller inflation balloon capacities, may achieve some benefits similar to reduce the total length of the catheter, reduce the balloon size as inflated inside the bladder, reduce the weight of the inflated balloon on the bladder, increase the patient's comfort and increase the drainage of the urine flow that of using a shorter length and smaller diameter balloon. This changes, however, requires changes in manufacturing processes.

The data suggest that an inflated balloon with a capacity of 10-15 ml, rather than the 30 ml capacity of the Foley catheters, most improves urine drainage and control the intra urethral leakage if it exists. This same capacity reduces the hydrostatic stress on the control valve by about 50% relative to a 30 ml inflation volume, and the balloon at lower inflation volumes has less hydrostatic pressure on the balloon material, allowing it to be more pliable when in the bladder and able to accept normal micturition contractions of the bladder without adverse effects on the inflation control valve and/or the balloon wall itself. These factors result in increased lifespan and durability of the catheter balloon in the bladder, increased patient comfort and reduced patient irritation lending itself to a higher degree of patient compliance with the indwelling catheter and, therefore, allow the patient to have a more rapid recovery.

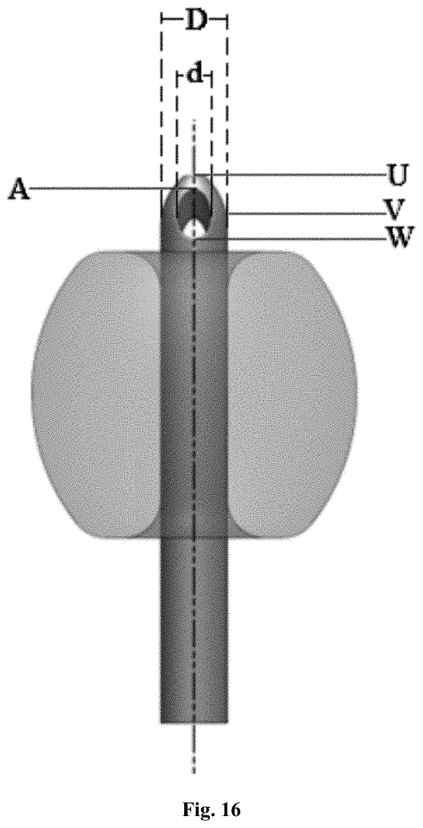

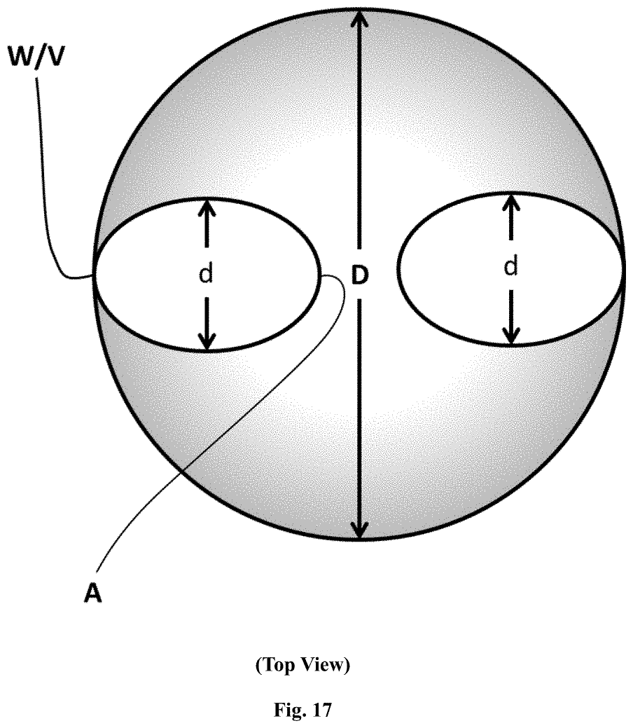

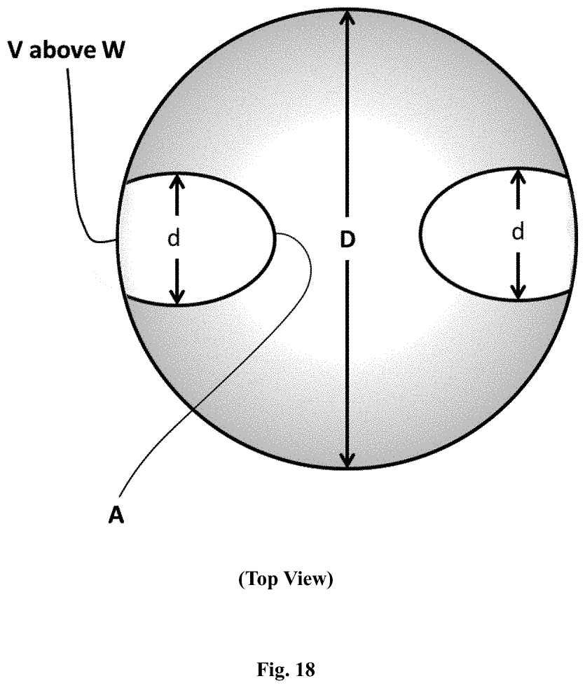

In some preferred embodiments of the present invention, the indwelling urinary catheter as shown in FIGS. 16-18 has a catheter body, a balloon, a balloon inflation lumen, a drainage lumen, and one or more (such as two) urine inflow openings at a distal end of the catheter body in fluid flow communication with the drainage lumen. For simple and clear illustration, the longitudinal axis of the catheter body is shown as a strictly straight line through the entire catheter body. The urine inflow openings have a distally facing bias with an opening orientation that is angularly displaced from being either parallel to or perpendicular to the longitudinal axis of the catheter body. The indwelling urinary catheter further includes a substantially frustoconical or conical distal tip, and a part of the urine inflow opening or the entire urine inflow opening pass through lateral wall surfaces of the distal tip, as will be explained in more details.

As shown in FIGS. 16-18, the catheter body has a diameter D (typically a constant diameter) before it is transitioned to (or extended to) the substantially frustoconical or conical distal tip at position “V”. Above the position “V”, the diameter of the frustoconical or conical distal tip is gradually decreased from D down to zero at the tip point (or pinnacle) “U.”

While the top position “A” of the urine inflow opening is typically located between “U” and “V”, the bottom position “W” of the urine inflow opening may be located below position “V”; at the same height or level of position “V”; or slightly above position “V.”

When the bottom position “W” of the urine inflow opening is located below position “V,” a part of the urine inflow opening passes through the lateral wall surfaces of the substantially frustoconical or conical distal tip. When the bottom position “W” of the urine inflow opening is located at the same height or level of, or above, position “V,” the entire urine inflow opening passes through the lateral wall surfaces of the substantially frustoconical or conical distal tip.

If measured along a measuring line that is perpendicular to the longitudinal axis of the catheter body as well as the longitudinal axis of the substantially frustoconical or conical distal tip, the urine inflow opening has a horizontal width that varies as the height of the measuring line varies. The horizontal width has a maximal value d at certain height, which is between the position “W” and the position “A.”

The height of the urine inflow opening Haw is defined as the height difference between position A and position W (for example 4 mm). The distance Duv is defined as the height difference between position U and position V. In particularly preferred embodiments, Haw is equal to or greater than 30% of Duv, 35% of Duv, 40% of Duv, 45% of Duv, 50% of Duv, 55% of Duv, 60% of Duv, 65% of Duv, or 70% of Duv. The maximal horizontal width d of the urine inflow opening is equal to or greater than 30% of catheter body diameter D, 35% of D, 40% of D, 45% of D, 50% of D, 55% of D, 60% of D, 65% of D, or 70% of D. The indwelling urinary catheter with these parameters can demonstrate numerous advantages over the prior art. For example, in the catheter as disclosed in Mikhail (U.S. Pat. No. 6,050,934) and Vega (U.S. Pat. No. 4,249,536), urine inflow ceases or dramatically slows down as soon its inflow openings pass into the bladder neck a short distance and are occluded by either the bladder neck tissue or the urethral tissue. In stark contrast, the distally biased orientation of the inflow openings in the inventive indwelling urinary catheter, causes the inflow openings to remain open and not become occluded by either the bladder neck or urethra as the catheter is being withdrawn even a relatively large distance, as compared to the known indwelling urinary catheter. In this manner, urine flow from the bladder and into the inventive indwelling urinary catheter remains continuous until either the muscles of the bladder or the bladder neck contract to seal off the bladder from the urethra.

Some of the prior arts are claiming that the taper end of the catheter will make the urine to continue to flow when the balloon inflated or even when the balloon deflated during the withdraw from the urethra, but they did not explain how that it is going to happened. In the above section there are more detail for this action.

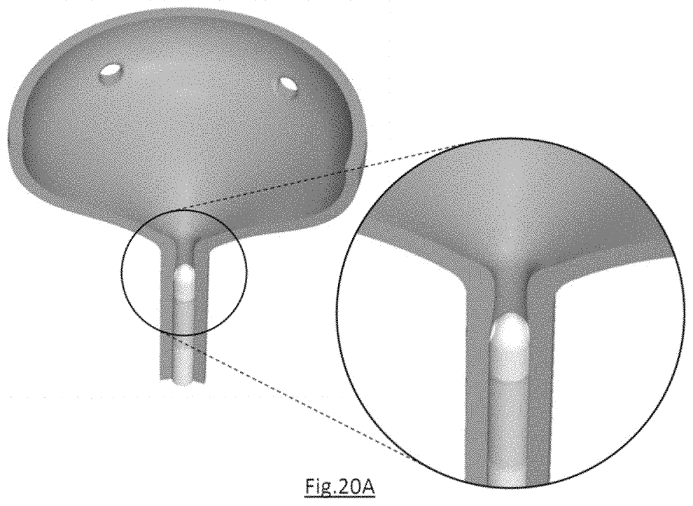

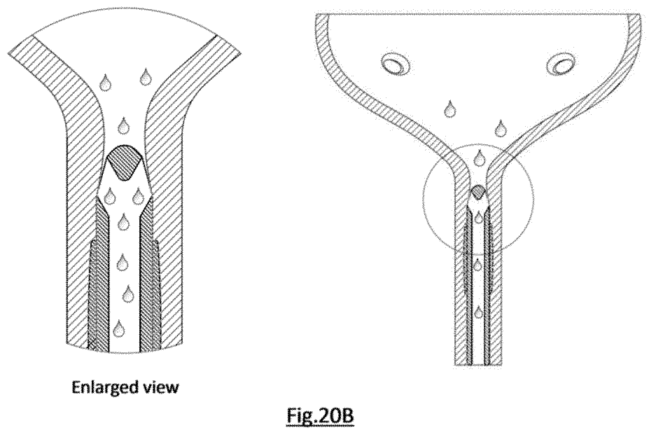

FIG. 20A is a 3D illustration that shows there is a gap between the urethra wall and the inflow openings that will allow a continues flow of urine while the catheter is traverse inside the urethra. and even with the top of the urethra is in a shrunk down condition. Due to the urethra's internal wall strength that will prevent any direct contact (adherence) between the urethra wall and the catheter tip at the zone of transition from small diameter (urethra) to a large diameter (catheter).

FIG. 20B (was FIG. 13 in the parent specification), it is added for the clarification of the urethra's behavior as the balloon was deflated and the catheter starts to traverse into the urethra. The urethra is a flexible tube is not a solid wall. In normal condition it has its own inside diameter which it is less than Catheter diameter. Diameter of the urethra can be expanded to allow the insertion of the catheter to reach the bladder in order to allow the drainage of the urine. When the balloon is deflated, the catheter will start to withdraw out from the bladder and inter the urethra. At that time the urethra will start to shrink down to its own diameter as long as there is none inside it to resist the shrinkage. Therefore, if the conical shape/taper end of the catheter inters the urethra, The shrinking wall of the urethra will adhere to the shape of the catheter as long as the diameter at this point is greater than the original inside diameter of the urethra. In this case if any of the inflow openings on the conical shape/taper end of the catheter will be covered and blocked and will not be able to drain any urine.

If the end of the catheter is tapered to a point that its diameter is smaller than the inside diameter of the urethra after being shrunk down, that condition may allow some urine to be drained. In this case, it will be required the design of the tip of the catheter to be able to accommodate the diameter of the drainage lumen (the size of the drainage opening) and two wall thicknesses of the catheter body.

In the new design, the inflow openings are on the top of the conical shape that will allow the drainage to be continued even when the urethra inside diameter is smaller than what is shown in FIG. 20B. Due to the shape of the transition zone from the urethra small diameter to a large diameter (the catheter outside diameter) and the internal wall strength factor of the urethra's wall, that will not be able to fully cover the two inflow openings, and will not allow for fully adherence between the urethra's wall and the top of the catheter, but it will create a gap between the urethra wall and the top of the catheter that will make the drainage to be continued.

Another situation, if the original diameter of the urethra is smaller than what is shown in FIG. 20B the urine still continues to flow, due to the oblate shape of the catheter top. In case of some urine is remain inside the urethra, at the action of contraction and or bladder spasms a pressure will be created that will cause the urethra wall to be expanded over the inflow openings and allow the urine to flow. In this case the catheter may be removed faster from the body. In the other design where the catheter has the inflow openings on the taper end of the catheter, the need to loosen the adherence between the urethra wall and the catheter may require more stronger bladder contraction and pressure.

While the present invention has been described with reference to specific exemplary embodiments, it will be evident that various modifications and changes may be made to these embodiments without departing from the broader spirit and scope of the invention as set forth in the claims. Accordingly, the specification and drawings are to be regarded in an illustrative rather than a restrictive sense.

1. An indwelling urinary catheter, which is a single unitary construct, having a catheter body, a balloon, a balloon inflation lumen, a drainage lumen, and one or more urine inflow openings at a distal end of the catheter body in fluid flow communication with the drainage lumen,
wherein the urine inflow openings have a distally facing bias with an opening orientation that is angularly displaced from being either parallel to or perpendicular to a longitudinal axis of the catheter body,
wherein the catheter further comprises a substantially frustoconical or conical distal tip, and the urine inflow openings pass through lateral wall surfaces of the distal tip;
wherein the catheter body is transitioned to (or extended to) the substantially frustoconical or conical distal tip at a position V;
wherein the urine inflow opening has a top position A and a bottom position W; and
wherein the bottom position W of the urine inflow opening is located below position V or at a same height or level of position V.

2. The indwelling urinary catheter of claim 1, wherein the tip point (or pinnacle) of said substantially frustoconical or conical distal tip is at position U;
wherein height of the urine inflow opening Haw is defined as the height difference between position A and position W;
wherein the distance Duv is defined as the height difference between position U and position V; and wherein Haw is equal to or greater than 30% of Duv, 35% of Duv, 40% of Duv, 45% of Duv, 50% of Duv, 55% of Duv, 60% of Duv, 65% of Duv, or 70% of Duv.

3. The indwelling urinary catheter of claim 2, wherein the catheter body has a diameter D at position V and/or W, and the urine inflow opening has a maximal horizontal width d; and
wherein d is equal to or greater than 30% of D, 35% of D, 40% of D, 45% of D, 50% of D, 55% of D, 60% of D, 65% of D, or 70% of D.

4. The indwelling urinary catheter of claim 1, wherein the urine inflow openings are in proximity to a distal end of the inflated balloon, and at least one of the urine inflow openings has a proximal end that is at the same level or blow the distal end of the inflated balloon.

5. The indwelling urinary catheter of claim 1, wherein the distally facing bias forms an angle á relative to a longitudinal axis of the catheter body 12 and angle á has a value between about 15 to about 75°.

6. The indwelling urinary catheter of claim 5, wherein angle á has a value between about 20 to about 45°; or a value between about 25 to about 35°.

7. The indwelling urinary catheter of claim 1, wherein the distal end of the catheter body further comprises a substantially frustoconical shaped distal tip and the urine inflow openings pass through lateral walls of the substantially frustoconical shaped distal tip and communicate with the drainage lumen in the catheter body.

8. The indwelling urinary catheter of claim 7, wherein the urine inflow openings further comprise at least two inflow openings positioned diametrically opposite each other about a circumference of the substantially frustoconical shaped distal tip.

9. The indwelling urinary catheter of claim 1, wherein a distance X represents a distance from a proximal end of an inflow opening to a distal end of the catheter body and is expressed by the equation:
X_{PI}=T_{PI}−S_{PI}; or X_{PI}=T_{PI}−(K+2L)
wherein, S_{PI} is distance for the present invention measured from a proximal end of the balloon to a proximal end of an inflow opening, T_{PI} is a distance for the present invesion measured from the proximal end of the balloon to the distal end the catheter body, K is the distance between two bonding lines, and L is the extended length of the balloon beyond each bonding line

10. The indwelling urinary catheter of claim 9, wherein S_{PI} is between about 23 to about 33 mm, and wherein T_{PI} is between about 30 to about 40 mm, depending on the balloon inflated volume.

11. The indwelling urinary catheter of claim 1, further comprising a third lumen in fluid flow communication with a port passing through a distal end of the catheter body.

12. The indwelling urinary catheter of claim 11, wherein the port passes through a distal tip of the catheter body.

13. The indwelling urinary catheter of claim 12, wherein the distal tip further comprises a generally frustoconical shape and the port passes through a lateral wall surface of the distal tip and is spaced apart from the urine inflow openings.

14. An indwelling urinary catheter, which is a single unitary construct, comprising a catheter body having a substantially conical distal tip, an inflatable balloon in proximity to the substantially conical distal tip, a balloon inflation lumen, a balloon inflation connector, a drainage lumen, a drainage port, and at least two urine inflow openings passing through lateral wall surfaces of the distal tip;
wherein the catheter body is transitioned to (or extended to) the substantially conical distal tip at a position V;
wherein the urine inflow openings have a top position A and a bottom position W; and
wherein the bottom position W of the urine inflow opening is located below position V or at a same height or level of position V.

15. The indwelling urinary catheter of claim 14, wherein each of the at least two urine inflow openings have an angle between about 20 to about 45° relative to a longitudinal axis of the catheter body; and wherein each of the at least two urine inflow openings have a substantially circular-shaped opening profile.

16. The indwelling urinary catheter of claim 15, wherein a proximal end of each of the at least two urine inflow openings are at the same level or blow a distal end of the inflatable balloon.

17. The indwelling urinary catheter of claim 14, wherein each of the at least two urine inflow openings have an angle between about 20 to about 45° relative to a longitudinal axis of the catheter body; and wherein the catheter further comprised a third lumen in the catheter body which is in fluid flow communication with a port passing through the substantially conical distal tip.

18. The indwelling urinary catheter of claim 17, wherein the port is positioned in spaced apart relationship from the at least two urine inflow openings.

19. The indwelling urinary catheter of claim 14, wherein the tip point (or pinnacle) of said substantially conical distal tip is at position U;
wherein height of the urine inflow opening Haw is defined as the height difference between position A and position W;
wherein the distance Duv is defined as the height difference between position U and position V; and
wherein Haw is equal to or greater than 30% of Duv, 35% of Duv, 40% of Duv, 45% of Duv, 50% of Duv, 55% of Duv, 60% of Duv, 65% of Duv, or 70% of Duv.

20. The indwelling urinary catheter of claim 19, wherein the catheter body has a diameter D at position V and/or W, and the urine inflow opening has a maximal horizontal width d; and
wherein d is equal to or greater than 30% of D, 35% of D, 40% of D, 45% of D, 50% of D, 55% of D, 60% of D, 65% of D, or 70% of D.

21. The indwelling urinary catheter of claim 14, wherein a distance between a proximal end of the balloon and a distal end of the catheter body (the insertion length of the catheter inside the bladder) is between 30 mm to about 40 mm based upon inflation capacity of the balloon.

22. The indwelling urinary catheter of claim 14, which obeys one or more of the following equations to determine the value of “Z” as the height of urine reduction inside the bladder:
Z=Y_{F}−M 1:
Z=S_{F}+L−M 2:
Z=Y_{F}−(T_{PI}−X_{PI}) 3:
Z=S_{F}+L−(T_{PI}−X_{PI}) 4:
wherein;
Z; is defined as the height of urine reduction inside the bladder
Y_{F}; is defined as the distance from the bottom of the each urine inflow opening to the proximal end of the inflated balloon in a standard Foley catheter
S_{F}; is defined as the distance between the bottom of the urine inflow opening to the proximal bonding balloon prior to balloon inflation in a standard Foley catheter
M; is defined as the height of the inflated balloon
L; is defined as the extended length of the balloon beyond each bonding line
K; is defined as the distance between two bonding lines of the balloon to the catheter
X_{PI}; is defined as the distance from proximal end of urine inflow opening to the distal end of the distal tip in the inventive catheter
and T_{PI}; is defined as the distance for the present invention taken from the proximal end of the balloon to the distal end of the catheter

23. The indwelling urinary catheter of claim 1, which is used for a method of reducing or eliminating an intra urethral leakage by a good interference between the balloon neck and the bladder neck by reducing the inflated balloon volume.

24. The indwelling urinary catheter of claim 1, wherein the urine inflow openings are in proximity to the distal end of the balloon when the balloon is in both balloon inflated state and balloon deflated state, and wherein angle á is 15° relative to the longitudinal axis of the catheter body, so as to maintain patency of the fluid flow into the drainage lumen as the balloon is deflated and catheter is withdrawn from the urethra.

25. The indwelling urinary catheter of claim 14, wherein each of the at least two urine inflow openings have a substantially circular-shaped opening profile, and wherein angle á is 20° relative to the longitudinal axis of the catheter body, so as to maintain patency of the fluid flow into the drainage lumen as the balloon is deflated and catheter is withdrawn from the urethra.

26. The indwelling urinary catheter of claim 14, further comprising a third lumen in the catheter body which is in fluid flow communication with a port passing through the substantially conical distal tip.

27. The indwelling urinary catheter of claim 21, further comprising a tertiary lumen configured to deliver or withdraw fluids from at least one of the urine inflow openings at the frustoconical distal tip, wherein the angle á is 15°.

28. The indwelling urinary catheter of claim 1, wherein the frustoconical distal tip is configured to remain substantially co-axial with the longitudinal axis of the catheter body when the balloon is in the inflated state.

29. The indwelling urinary catheter of claim 1, wherein a distance between the distal end of the balloon in the inflated state and the proximal end of the urinary inflow openings is between about 0 mm to about 4 mm.
